(19) United States
(12) Patent Application Publication
(10) Pub. No.: US 2020/0294638 A1
(43) Pub. Date: Sep. 17, 2020

(54) MOBILE INFORMATION TERMINAL, PHYSIOLOGICAL INFORMATION MANAGEMENT METHOD, AND NON-TRANSITORY COMPUTER READABLE STORAGE MEDIUM

(71) Applicant: NIHON KOHDEN CORPORATION, Tokyo (JP)

(72) Inventors: Norihito KONNO, Tokyo (JP); Fumiyuki MATSUMURA, Tokyo (JP)

(21) Appl. No.: 16/807,572

(22) Filed: Mar. 3, 2020

(30) Foreign Application Priority Data
Mar. 15, 2019 (JP) 2019-048506

(51) Int. Cl. G16H 10/65; H04W 12/00; A61B 5/00; A61B 5/117

(52) U.S. Cl. CPC G16H 10/65; A61B 5/117; A61B 5/6801; H04W 12/0051

(57) ABSTRACT

A mobile information terminal includes: a storage storing sensor identification information and patient identification information, while associating with each other; a wireless communication interface receiving a connection request signal; and a processor, when the wireless communication interface receives the connection request signal from the sensor identified by the sensor identification information, determining whether the sensor identification information is associated with the patient identification information or not, and the processor denying establishment of a wireless connection with the sensor that is subjected to the determination, in a case where the sensor identification information that is subjected to the determination is not associated with the patient identification information, and establishing a wireless connection with the sensor that is subjected to the determination, in a case where the sensor identification information that is subjected to the determination is associated with the patient identification information.

### CROSS-REFERENCE TO RELATED APPLICATIONS

This application is based upon and claims the benefit of priority from prior Japanese patent application No. 2019-048506, filed on Mar. 15, 2019, the entire contents of which are incorporated herein by reference.

### TECHNICAL FIELD

The presently disclosed subject matter relates to a mobile information terminal and physiological information management method for processing physiological information of a patient, a computer program that causes the mobile information terminal to execute the physiological information management method, and a non-transitory computer readable storage medium that stores the computer program.

### BACKGROUND ART

JP-A-2017-086664 discloses a physiological information measurement system including a sensor and a display device. The sensor is attached to a patient to acquire physiological information of the patient. The sensor is wiredly connected to the display device. The sensor transmits a signal corresponding to the acquired physiological information, to the display device. The display device displays the physiological information of the patient corresponding to the received signal.

JP-A-2005-168600 discloses a physiological information display system including a sensor, a wireless transmitter, and a display device. The sensor is attached to a patient to acquire physiological information of the patient. The sensor is wiredly connected to the wireless transmitter. The wireless transmitter wirelessly transmits a signal corresponding to the acquired physiological information, to the display device. The display device displays the physiological information of the patient corresponding to the received signal.

From the viewpoints of reduction of the weight of a device, and improvement of the degree of freedom of movement of a patient, it is desired to configure the wired connection portion that exists between the sensor and the display device, as a completely wireless portion. However, the wired connection may function as means for visually checking association between the patient to whom the sensor is attached, and the display device or the wireless transmitter.

In the configuration disclosed in JP-A-2017-086664, for example, the sensor and the display device are directly wiredly connected to each other. In this case, the fact that the physiological information displayed on the display device is information of the patient to whom the sensor is attached is clear at a glance. When the connection between the sensor and the display device is configured with a wireless connection, a careful preliminary checking work must be performed in order to make sure that the patient to whom the sensor is attached and the displayed physiological information are associated with each other.

In the configuration disclosed in JP-A-2005-168600, the degree of freedom of movement with respect to the display device is ensured. However, the patient is required to carry the wireless transmitter. When the connection between the sensor and the wireless transmitter is configured with a wireless connection, a careful preliminary work of checking that both the devices are associated with the same patient is necessary.

It is an object of the presently disclosed subject matter to configure a sensor with a wireless connection, to facilitate management of physiological information while improving the degree of freedom of movement of a patient.

### SUMMARY OF INVENTION

According to an aspect of the invention, there is provided a mobile information terminal comprising: a storage that is configured to store sensor identification information for identifying a sensor which is attached to a patient and which wirelessly transmits a sensor signal corresponding to physiological information of the patient, and patient identification information for identifying the patient, while associating with each other; a wireless communication interface that is configured to receive a connection request signal from at least one sensor, and that is configured to receive the sensor signal from a sensor with which a wireless connection is established; and a processor that, when the wireless communication interface receives the connection request signal from the sensor identified by the sensor identification information stored in the storage, is configured to determine whether the sensor identification information is associated with the patient identification information or not, and the processor that is configured to deny establishment of a wireless connection with the sensor that is subjected to the determination, in a case where the sensor identification information that is subjected to the determination is not associated with the patient identification information, and establish a wireless connection with the sensor that is subjected to the determination, in a case where the sensor identification information that is subjected to the determination is associated with the patient identification information.

### BRIEF DESCRIPTION OF DRAWINGS

FIG. 1 exemplarily illustrates the functional configuration of a mobile information terminal of an embodiment.

FIG. 2 illustrates association between sensor identification information and patient identification information.

FIG. 3 exemplarily illustrates the flow of processes performed by the mobile information terminal.

FIG. 4 exemplarily illustrates the flow of processes performed by the mobile information terminal.

FIG. 5 illustrates the operation of the mobile information terminal.

FIG. 6 exemplarily illustrates the flow of processes performed by the mobile information terminal.

### DESCRIPTION OF EMBODIMENTS

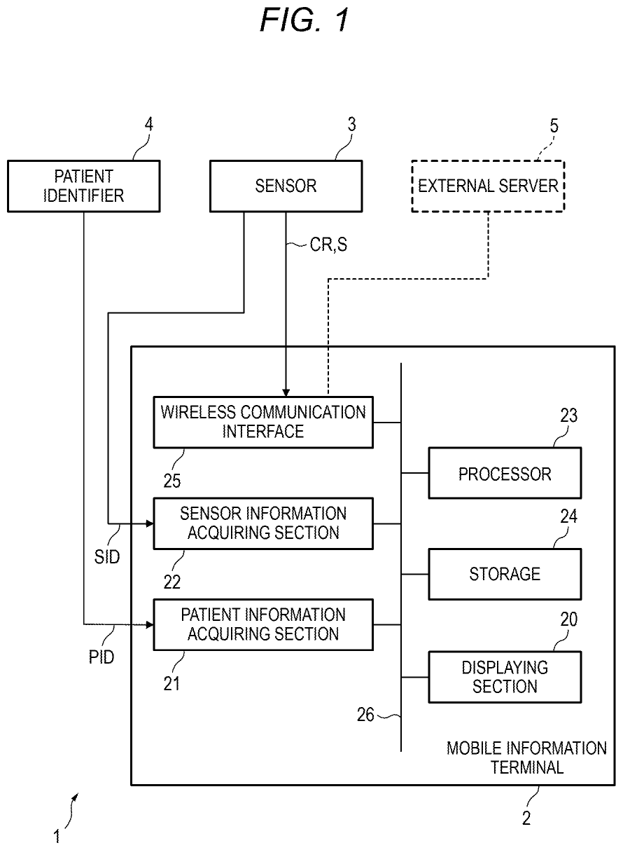

Hereinafter, an embodiment will be described in detail with reference to the accompanying drawings. FIG. 1 exemplarily illustrates the functional configuration of a physiological information management system 1 of the embodiment.

The physiological information management system 1 is a system that manages physiological information of patients. The physiological information includes values of various physiological parameters. Examples of the physiological parameters are the body temperature, the blood pressure, an electrocardiogram, brain waves, and the arterial oxygen saturation (SpO2). The physiological information management system 1 includes a mobile information terminal 2, sensors 3, and patient identifiers 4.

The mobile information terminal 2 is a portable device having a displaying function and an information processing function. Examples of the mobile information terminal 2 are a smartphone, a tablet terminal, and a head-mounted display. The configuration of the mobile information terminal 2 will be described in detail later.

Each of the sensors 3 is a device that is to be attached to a patient to acquire physiological information of the patient, and that wirelessly transmits a sensor signal S corresponding to the physiological information. Each of the sensors 3 includes a sensor identifier. The sensor identifier holds sensor identification information SID identifying the individual sensor. The sensor identification information SID may be provided on a package of the sensor or the sensor body in the form of image data such as a text or bar code that indicates the manufacturing number, or in the form of non-image data that are to be transmitted through non-contact communication.

Each of the patient identifiers 4 is an article that holds patient identification information PID identifying the corresponding individual patient. Examples of the patient identifier 4 are a wrist band that is to be attached to the arm of the patient, a name tag that is to be attached to the cloth of the patient, and a nameplate that is to be attached to a bed or medical ward in which the patient stays. The patient identification information PID may be provided in the form of image data such as a text or bar code that indicates the name or number of the patient, or in the form of non-image data that are to be transmitted through non-contact communication.

The mobile information terminal 2 includes a displaying section 20, a patient information acquiring section 21, a sensor information acquiring section 22, a processor 23, a storage 24, a wireless communication interface 25, and a communication bus 26. The displaying section 20, the patient information acquiring section 21, the sensor information acquiring section 22, the processor 23, the storage 24, and the wireless communication interface 25 are mutually communicably connected to one another through the communication bus 26.

The displaying section 20 is configured so as to be able to display various kinds of information thereon. Examples of the displaying section 20 are a liquid crystal display device and an organic EL display device. The displaying section 20 may include a touch panel device through which a touch input is enabled.

The patient information acquiring section 21 is configured so as to acquire patient identification information PID from the patient identifiers 4. In the case where the patient identification information PID is provided in the form of image information such as a text or a bar code, the patient information acquiring section 21 may be configured so as to include a camera that can acquire the image information. In the case where the patient identification information PID is provided in the form of non-image data, the patient information acquiring section 21 may be configured so as to include a device that can perform wireless near field communication (NFC) or wireless Bluetooth (registered trademark) communication through which the non-image data can be acquired in a non-contact manner.

The sensor information acquiring section 22 is configured so as to acquire sensor identification information SID from the sensors 3. In the case where the sensor identification information SID is provided in the form of image information such as a text or a bar code, the sensor information acquiring section 22 may be configured so as to include a camera which can acquire the image information. In the case where the sensor identification information SID is provided in the form of non-image data, the sensor information acquiring section 22 may be configured so as to include a device that can perform wireless near field communication (NFC) or wireless Bluetooth (registered trademark) communication through which the non-image data can be acquired in a non-contact manner.

The functions of the patient information acquiring section 21 and the sensor information acquiring section 22 may be realized by a common device, or by individual devices.

The processor 23 is configured so as to perform the pairing that enables wireless communication between the mobile information terminal 2 and the sensor 3. Specifically, the sensor identification information SID that is acquired by the sensor information acquiring section 22 is stored in the storage 24. The mobile information terminal 2 is configured so as to enable the sensor signal S transmitted from the sensor 3 that has the sensor identification information SID stored in the storage 24, to be received by the wireless communication interface 25.

The wireless communication interface 25 may include an adequate signal converting circuit that converts the received signal S to a form on which processing can be performed by the processor 23.

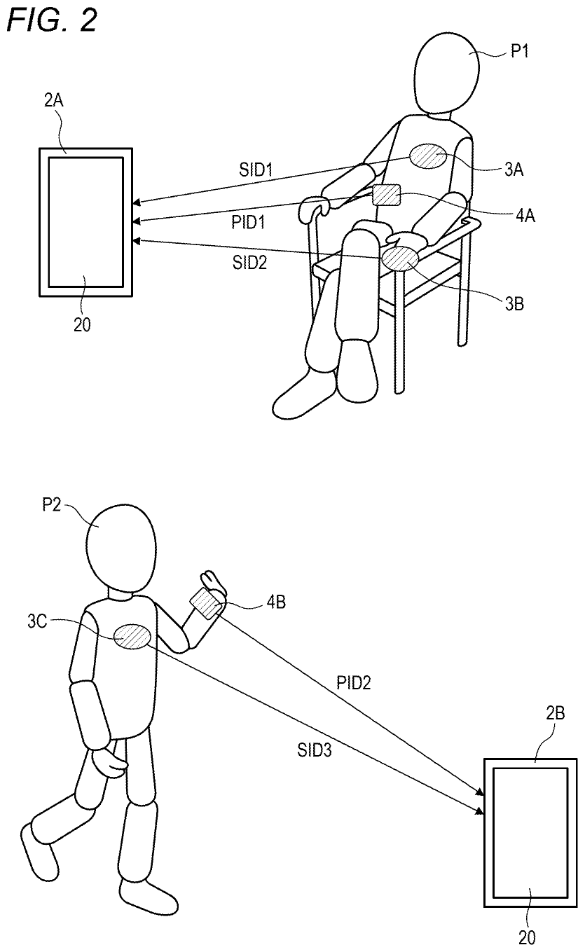

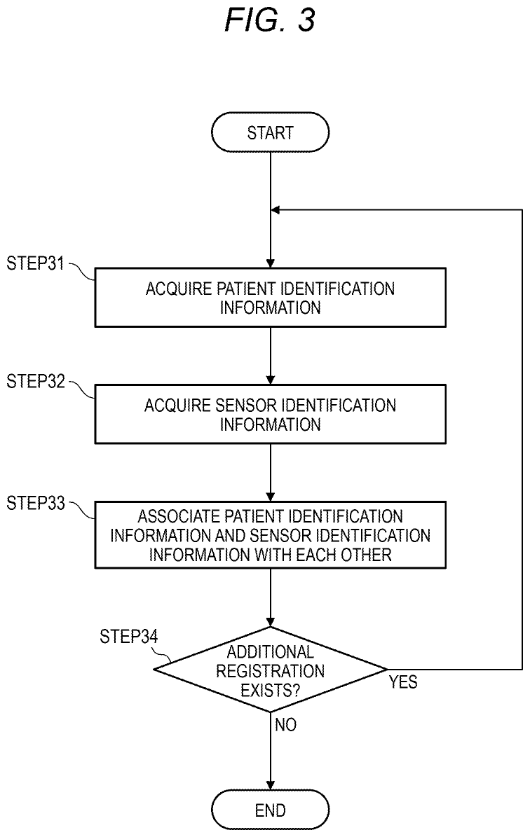

The processor 3 is configured so as to perform a process of associating the patient identification information PID acquired by the patient information acquiring section 21 with the sensor identification information SID acquired by the sensor information acquiring section 22. An example of the process will be described in detail with reference to FIGS. 2 and 3.

FIG. 2 illustrates, as the mobile information terminal 2, a first mobile information terminal 2A and a second the mobile information terminal 2B. The first mobile information terminal 2A is allocated to a first patient P1, and used for managing physiological information of the first patient P1. The second the mobile information terminal 2B is allocated to a second patient P2, and used for managing physiological information of the second patient P2.

In FIG. 2, a first sensor 3A, a second sensor 3B, and a third sensor 3C are illustrated as the sensors 3. The first sensor 3A is attached to the chest of the first patient P1. The second sensor 3B is attached to the finger tip of the first patient P1. The third sensor 3C is attached to the chest of the second patient P2. The first sensor 3A and the third sensor 3C are, for example, electrodes for acquiring an electrocardiogram. The second sensor 3B is, for example, a probe for acquiring the arterial oxygen saturation.

In FIG. 2, a first patient identifier 4A and a second patient identifier 4B are illustrated as the patient identifiers 4. The first patient identifier 4A is attached to the cloth of the first patient P1 in the form of a name tag. The second patient identifier 4B is attached to the arm of the second patient P2 in the form of a wrist band.

Referring to FIG. 3, the process of associating the patient identification information PID with the sensor identification information SID that is performed in the first mobile information terminal 2A will be described.

First, the patient identification information PID is acquired by the patient information acquiring section 21 (STEP 31). For example, the processor 23 causes a message for prompting the user to acquire the patient identification information PID, to be displayed on the displaying section 20. In the example illustrated in FIG. 2, by using the patient information acquiring section 21, the user acquires patient identification information PID1 of the first patient P1 from the first patient identifier 4A of the first patient P1.

Then, the sensor identification information SID is acquired by the sensor information acquiring section 22 (STEP 32). For example, the processor 23 causes a message for prompting the user to acquire the sensor identification information SID, to be displayed on the displaying section 20. In the example illustrated in FIG. 2, by using the sensor information acquiring section 22, the user acquires sensor identification information SID1 of the first sensor 3A attached to the first patient P1. In the case where the wireless communication between the mobile information terminal 2 and the sensors 3 is performed based on communication standard such as BLE (Bluetooth Low Energy), the process corresponds to the pairing.

In the case where the wireless communication between the mobile information terminal 2 and the sensors 3 is performed based on a communication standard such as BLE (Bluetooth Low Energy), the sensor identification information SID that is once stored in the storage 24 through the pairing is in principle maintained without being erased.

The order of the acquisition of the patient identification information (STEP 31) and that of the sensor identification information (STEP 32) may be reversed.

When the fact that the acquisition of the patient identification information, and that of the sensor identification information are completed is notified to the first mobile information terminal 2A by the user, the patient identification information PID and the sensor identification information SID are associated with each other by the processor 23 (STEP 33). The association between sets of identification information can be performed by a well-known technique. The notification to the first mobile information terminal 2A may be performed by an operation on a predetermined button (including that displayed on the displaying section 20) or a voice input.

In the embodiment, the processor 23 associates the patient identification information PID1 acquired from the first patient identifier 4A, with the sensor identification information SIDI acquired from the first sensor 3A, and then stores these sets of information in the storage 24. That is, information indicating the fact that the first sensor 3A is attached to the first patient P1 is stored in the storage 24.

Then, it is checked whether additional registration exists or not (STEP 34). For example, the processor 23 causes a message for checking whether additional registration exists or not, to be displayed on the displaying section 20. In the example illustrated in FIG. 2, also the second sensor 3B is attached to the first patient P1. Therefore, the user inputs information indicating that additional registration is necessary, in the first mobile information terminal 2A (YES in STEP 34).

In this case, the acquisition of the patient identification information PID (STEP 31), that of the sensor identification information SID (STEP 32), and association of the patient identification information PID and the sensor identification information SID (STEP 33) that are described above are repeated. Since one patient identification information PID is allocated to one mobile information terminal 2, reacquisition of the patient identification information PID may be omitted.

In the example illustrated in FIG. 2, the patient identification information PID1 acquired from the first patient identifier 4A is associated with the sensor identification information SID2 acquired from the second sensor 3B, and then these sets of information are stored in the storage 24. That is, information indicating the fact that the second sensor 3B is attached to the first patient P1 is stored in the storage 24.

Then, it is checked whether additional registration exists or not (STEP 34). In the example illustrated in FIG. 2, the sensor 3 that is required to be additionally registered with respect to the first patient P1 does not exists. Therefore, the user inputs information indicating that additional registration is not necessary, in the first mobile information terminal 2A (NO in STEP 34). Then, the process is ended.

Also in the second mobile information terminal 2B that is allocated to the second patient P2, the same or similar process is executed. In this case, the patient identification information PID2 acquired from the second patient identifier 4B is associated with the sensor identification information SID3 acquired from the third sensor 3C, and then these sets of information are stored in the storage 24. That is, information indicating the fact that the third sensor 3C is attached to the second patient P2 is stored in the storage 24.

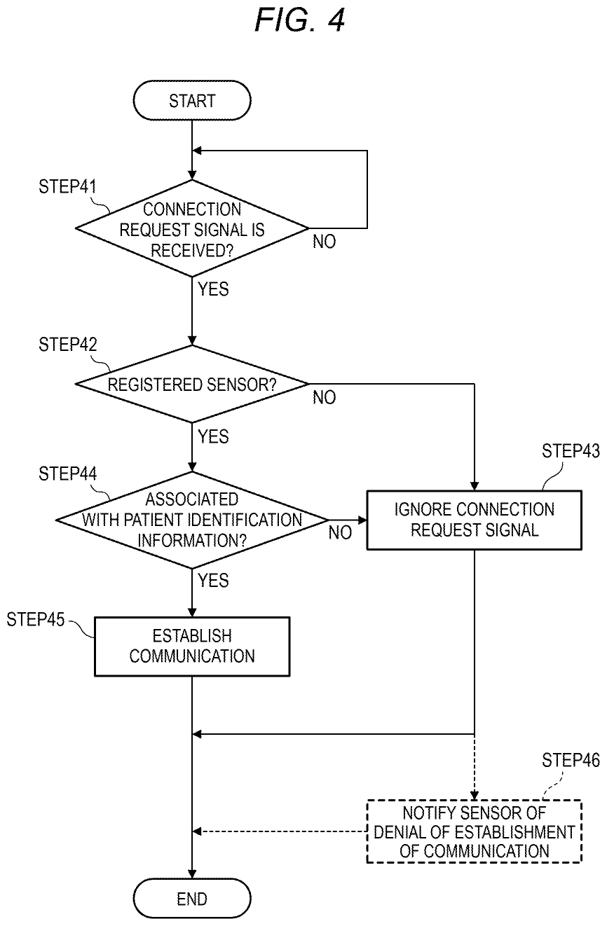

Referring to FIG. 4, next, an example of a process of establishing communication with the sensors 3 that is performed in the mobile information terminal 2 will be described.

Each of the sensors 3 is configured so as to wirelessly transmit a connection request signal CR. The connection request signal CR contains the sensor identification information SID of the sensor 3, or information corresponding to the sensor identification information. The processor 23 of the mobile information terminal 2 determines whether the wireless communication interface 25 receives the connection request signal CR or not (STEP 41). This process is repeated until the reception of the connection request signal CR is confirmed (NO in STEP 41).

If it is determined that the wireless communication interface 25 receives the connection request signal CR (YES in STEP 41), the processor 23 determines whether the sensor 3 that transmits the connection request signal CR already establishes the pairing or not (STEP 42). Specifically, the processor 23 determines whether the sensor identification information SID corresponding to the received connection request signal CR is stored in the storage 24 or not.

The case where the sensor identification information SID corresponding to the received connection request signal CR is not stored in the storage 24 (NO in STEP 42) means that the pairing which has been described with reference to STEP 32 of FIG. 3 is not established with respect to the sensor 3 transmitting the connection request signal CR. In this case, the processor 23 denies establishment of wireless communication with the sensor 3 transmitting the connection request signal CR (STEP 43). For example, the processor 23 ignores the connection request signal CR.

If the sensor identification information SID corresponding to the received connection request signal CR is stored in the storage 24 (YES in STEP 42), the processor 23 determines whether the sensor identification information SID is stored in the storage 24 in the state where the information is associated with the patient identification information PID or not (STEP 44).

If it is determined that the sensor identification information SID corresponding to the received connection request signal CR is associated with the patient identification information PID (YES in STEP 44), the processor 23 permits establishment of wireless communication with the sensor 3 that transmits the connection request signal CR (STEP 45). The sensor 3 starts wireless transmission of the sensor signal S corresponding to the acquired physiological information, and the wireless communication interface 25 is enabled to receive the sensor signal S.

For example, physiological information is displayed on the displaying section 20 of the mobile information terminal 2. The physiological information corresponding to the sensor signal S that is received by the wireless communication interface 25 is visualized through adequate processes, and then displayed on the displaying section 20. In the example illustrated in FIG. 2, an electrocardiogram that is acquired by the first sensor 3A, and the arterial oxygen saturation that is acquired by the second sensor 3B are displayed as physiological information of the first patient P1 on the displaying section 20 of the first mobile information terminal 2A. In the same or similar manner, an electrocardiogram that is acquired by the third sensor 3C is displayed as physiological information of the second patient P2 on the displaying section 20 of the second mobile information terminal 2B.

On the other hand, if it is determined that the sensor identification information SID corresponding to the received connection request signal CR is not associated with the patient identification information PID (NO in STEP 44), the processor 23 denies establishment of wireless communication with the sensor 3 that transmits the connection request signal CR (STEP 43). For example, the processor 23 ignores the connection request signal CR.

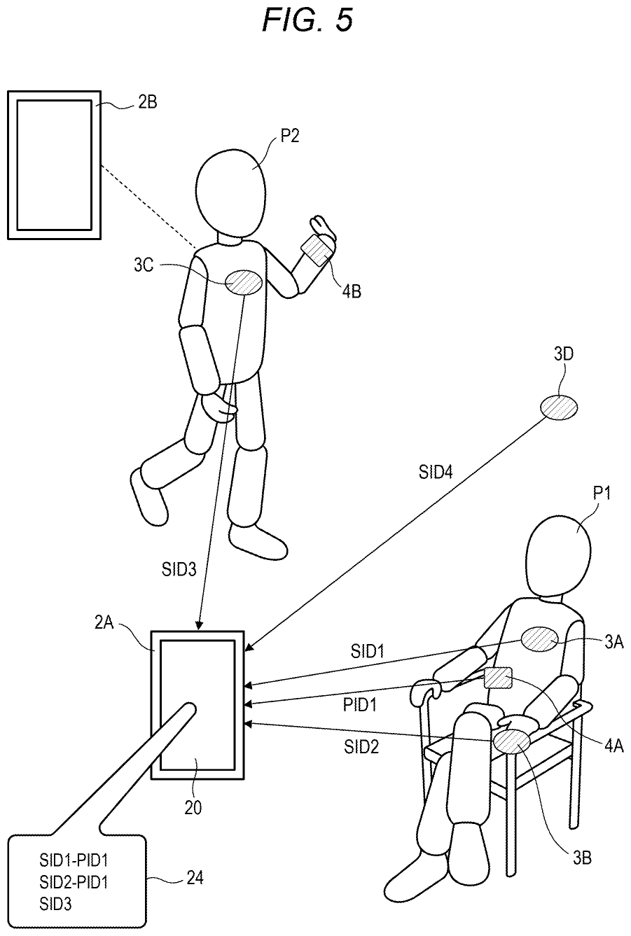

Referring to FIG. 5, advantages of the above-described configuration will be described. In the embodiment, the first mobile information terminal 2A is allocated to the first patient P1. The first sensor 3A and the second sensor 3B are attached to the first patient P1. Each of the sensor identification information SIDI of the first sensor 3A, and the sensor identification information SID2 of the second sensor 3B is stored in the storage 24 of the first mobile information terminal 2A while being associated with the patient identification information PID1 of the first patient P1. The sensor identification information SID3 of the third sensor 3C in which pairing has been established in the past is stored in the storage 24 of the first mobile information terminal 2A without being erased.

The wireless communication interface 25 of the first mobile information terminal 2A receives also a connection request signal CR of a fourth sensor 3D that is not attached to the first patient P1. However, sensor identification information SID4 for identifying the fourth sensor 3D is not stored in the storage 24. Therefore, the connection request signal CR that is transmitted from the fourth sensor 3D is ignored (NO in STEP 42 of FIG. 4).

The third sensor 3C is attached to the second patient P2 to whom the second mobile information terminal 2B is allocated. When the second patient P2 separates by a certain distance from the second mobile information terminal 2B, the wireless communication that has been established between the third sensor 3C and the second mobile information terminal 2B is interrupted. In FIG. 5, the state where the wireless communication is interrupted is indicated by the broken line.

When, in this state, the second patient P2 approaches the first patient P1, the connection request signal CR that is transmitted from the third sensor 3C is received by the wireless communication interface 25 of the first mobile information terminal 2A. However, the sensor identification information SID3 of the third sensor 3C that is stored in the storage 24 is not associated with specific patient identification information PID, and therefore the connection request signal CR that is transmitted from the third sensor 3C is ignored (NO in STEP 44 of FIG. 4).

A situation is assumed where the first mobile information terminal 2A does not have the above-described configuration, and operates according to specification where wireless communication is automatically established between devices that have undergone pairing in the past. In the case, as in the embodiment, the sensor identification information SID3 of the third sensor 3C is stored in the storage 24 without being erased, the first mobile information terminal 2A tries to establish wireless communication with the third sensor 3C. In the case where the third sensor 3C operates according to specification where the third sensor communicates only with a mobile information terminal with which pairing is established, when pairing is established between the first mobile information terminal 2A and the third sensor 3C, acquisition of physiological information from the third sensor 3C that is to be originally performed in the second mobile information terminal 2B is not performed, and that of physiological information from the third sensor 3C that should not be originally performed is performed by the first mobile information terminal 2A.

In other words, when the mobile information terminal 2 has the above-described configuration, only physiological information based on the sensor signal S that is transmitted from the sensor 3 which is identified by the sensor identification information SID associated with the patient identification information PID is acquired by the mobile information terminal 2. Even when a visual check of a wired connection is not performed, therefore, a conviction that an erroneous connection between the mobile information terminal 2 and the sensor 3 is avoided is easily obtained. Under a situation where, after a certain patient leaves the bed, the mobile information terminal 2 and the sensor 3 can be reused for another patient, particularly, it is very advantageous to obtain a conviction that an erroneous connection is avoided. When the sensor 3 is configured with a wireless connection, therefore, management of physiological information can be facilitated while improving the degree of freedom of movement of the patient.

As a response to the connection request signal CR from the sensor 3 in which establishment of a wireless communication is to be denied, the processor 23 may causes the wireless communication interface 25 to transmit a signal notifying that establishment of communication is denied, to the sensor 3 (STEP 46 of FIG. 4). In the case where the sensor 3 has a configuration where the sensor receives such a notification, and then informs the user of the notification, the fact that the wireless communication with the mobile information terminal 2 cannot be established can be easily known, and the convenience of the user is improved.

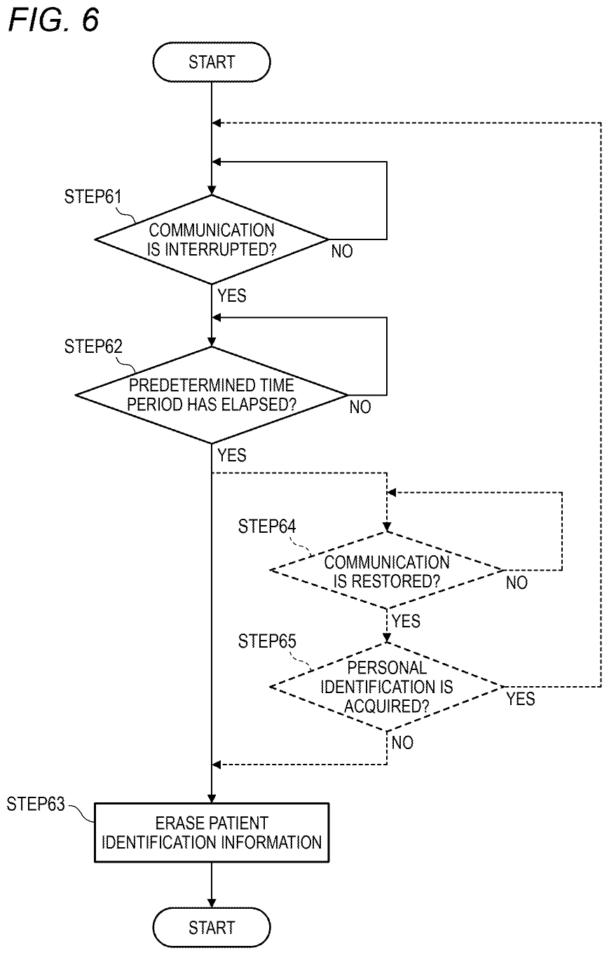

FIG. 6 exemplarily illustrates processes that are performed when the wireless connection between the mobile information terminal 2 and the sensor 3 is interrupted for any reason.

The processor 23 determines whether the wireless communication between the mobile information terminal 2 and the sensor 3 is interrupted or not (STEP 61). For example, the determination may be conducted by monitoring the signal level of the sensor signal S that is received by the wireless communication interface 25. When the signal level is lowered to a predetermined value, it can be determined that the wireless communication is interrupted.

If it is determined that the wireless communication is interrupted (YES in STEP 61), the processor 23 determines whether a predetermined time period has elapsed from the interruption of the communication or not (STEP 62). The determination may be conducted by a well-known technique using an internal timer or the like. For example, the predetermined time period may be 30 minutes.

If it is determined that the predetermined time period has elapsed from the interruption of the communication (YES in STEP 62), the processor 23 erases the patient identification information PID that is stored in the storage 24 (STEP 63). That is, the association with the sensor identification information SID stored in the storage 24 is canceled.

Even when the mobile information terminal 2 thereafter receives the connection request signal CR from any one of the sensors 3, sensor identification information that is associated with the patient identification information PID does not exist in the storage 24, and therefore the mobile information terminal 2 does not establish a wireless connection with any one of the sensors 3 (NO in STEP 44 of FIG. 4).

According to the configuration, even when, after the wireless connection with the sensor 3 is interrupted for any reason, the mobile information terminal returns to the state where communication is enabled, it is possible to avoid a situation where the mobile information terminal 2 establishes wireless communication with an unintentional one of the sensors 3.

In this case, the patient identification information PID and the sensor identification information SID are again associated with each other (STEP 33 of FIG. 3).

After it is determined that the predetermined time period has elapsed from the interruption of the communication (YES in STEP 62), alternatively, the processor 23 may determine whether wireless communication with the sensor 3 is restored or not (STEP 64). For example, also the determination may be conducted by monitoring the signal level of the sensor signal S that is received by the wireless communication interface 25. When the signal level is raised to a predetermined value, it can be determined that the wireless communication is restored.

If it is determined that the wireless communication with the sensor 3 is restored (YES in STEP 64), the processor 23 of the mobile information terminal 2 conducts a process of identifying a person (STEP 65). In the example illustrated in FIG. 5, the first mobile information terminal 2A identifies whether the patient to whom the terminal itself is allocated is the first patient P1 or not. For example, a confirmation message of “Are you first patient P1?” is displayed on the displaying section 20. The person who gets the confirmation inputs a response of right or wrong by means of an adequate technique.

If it is determined that the personal identification is acquired (YES in STEP 65), the processor 23 returns the process to STEP 61 without erasing the patient identification information PID stored in the storage 24. Namely, the association of the patient identification information PID and the sensor identification information SID is maintained. Therefore, the wireless communication between the mobile information terminal 2 and the sensor 3 is restarted.

When the mobile information terminal 2 has been already reused, and allocated to another patient, for example, the personal identification is mot acquired (NO in STEP 65). In this case, the processor 23 erases the patient identification information PID stored in the storage 24 (STEP 63). The process of association is again performed between the patient identification information PID corresponding to the other patient, and the sensor identification information SID of the sensor 3 attached to the other patient.

According to the configuration, the frequency of the process of, in the case where the wireless connection with the sensor 3 is interrupted for any reason, reassociating the patient identification information PID and the sensor identification information SID can be lowered. Therefore, the convenience of a patient can be improved while facilitating the management of physiological information.

Alternatively, the mobile information terminal 2 may enable the processor 23 to receive instructions for erasing the patient identification information PID from the storage 24. Namely, the association of the patient identification information PID and the sensor identification information SID can be forcibly canceled. When the user operates a user interface that is not illustrated, for example, the instructions may be input. Alternatively, an erasure instruction signal that is transmitted from a dedicated wireless communication device may be received by the wireless communication interface 25.

According to the configuration, an operation is enabled in which, when a patient leaves the bed, for example, such an erasing process is performed. Therefore, it is possible to easily avoid a situation where the patient identification information PID of the previous patient remains in the storage 24 of the mobile information terminal 2 that is allocated to another patient.

The above-described function of the processor 23 may be realized by a general-purpose microprocessor which operates in cooperation with a general-purpose memory. Examples of such a general-purpose microprocessor are a CPU, an MPU, and a GPU. Examples of such a general-purpose memory are a ROM and a RAM. A ROM may store computer programs that execute the above-described processes. A ROM is an example of the storage medium that stores the computer programs. The processor 23 designates at least part of the programs stored in the ROM, loads the designated programs into the RAM, and executes the above-described processes in cooperation with the RAM. The storage 24 may be used as the above-described general-purpose memory. The processor 23 may be realized by a dedicated integrated circuit such as a microcontroller, ASIC, or FPGA that can execute computer programs for realizing the above-described processes. In this case, the storage 24 may be packaged in the same device as the processor 23. The processor 23 may be realized by a combination of a general-purpose microprocessor and a dedicated integrated circuit.

As illustrated in FIG. 1, the mobile information terminal 2 can be configured so as be communicable with an external server 5 through a communication network that is not illustrated. In this case, the computer programs that execute the above-described processes can be downloaded from the external server 5 through the network. In this case, the external server 5 is an example of the storage medium that stores the computer programs. Alternatively, at least part of the above-described physiological information management method may be executed by a processor of the external server 5.

The above-described embodiment is a mere example for facilitating understanding of the presently disclosed subject matter. The configuration of the embodiment may be adequately changed or improved without departing from the spirit of the presently disclosed subject matter.

In the above-described embodiment, the patient identification information PID is erased from the storage 24 in order to cancel the association between the patient identification information PID and the sensor identification information SID. However, the patient identification information PID is not necessarily erased from the storage 24. For example, a flag indicating whether association with the sensor identification information SID is performed or not may be provided to the patient identification information PID. When the value of the flag is changed, the association between the patient identification information PID and the sensor identification information SID can be canceled.

In the embodiment, physiological information corresponding the sensor signal S transmitted from the sensor 3 is displayed on the displaying section 20 of the mobile information terminal 2. However, the physiological information may be displayed on a display device that is different from the mobile information terminal 2.

According to the above-described configuration, only physiological information based on the sensor signal that is transmitted from the sensor which is identified by the sensor identification information associated with the patient identification information is acquired by the mobile information terminal. Even when a visual check of a wired connection is not performed, therefore, it is possible to easily receive assurance that erroneous connection between the mobile information terminal and the sensor is avoided. Under the situation where, after a certain patient leaves the bed, the mobile information terminal and the sensor can be reused for another patient, particularly, it is very advantageous to receive assurance that an erroneous connection is avoided. When the sensor is configured with a wireless connection, therefore, management of physiological information can be facilitated while improving the degree of freedom of movement of the patient.

What is claimed is:

1. A mobile information terminal comprising:
a storage that is configured to store sensor identification information for identifying a sensor which is attached to a patient and which wirelessly transmits a sensor signal corresponding to physiological information of the patient, and patient identification information for identifying the patient, while associating with each other;
a wireless communication interface that is configured to receive a connection request signal from at least one sensor, and that is configured to receive the sensor signal from a sensor with which a wireless connection is established; and
a processor that, when the wireless communication interface receives the connection request signal from the sensor identified by the sensor identification information stored in the storage, is configured to determine whether the sensor identification information is associated with the patient identification information or not, and
the processor that is configured to
deny establishment of a wireless connection with the sensor that is subjected to the determination, in a case where the sensor identification information that is subjected to the determination is not associated with the patient identification information, and
establish a wireless connection with the sensor that is subjected to the determination, in a case where the sensor identification information that is subjected to the determination is associated with the patient identification information.

2. The mobile information terminal according to claim 1, wherein, in a case where the wireless connection is interrupted for a predetermined time period or longer, the processor is configured to cancel the association between the patient identification information and the sensor identification information.

3. The mobile information terminal according to claim 1, wherein,
in a case where, after the wireless connection is interrupted for a predetermined time period or longer, a communication enabled state is restored,
the processor is configured to
check whether a user of the mobile information terminal is the patient who is identified by the patient identification information or not, and,
if it is determined that the user of the mobile information terminal is the patient who is identified by the patient identification information, maintain the association between the patient identification information and the sensor identification information.

4. The mobile information terminal according to claim 1, wherein the mobile information terminal can receive an instruction for causing the processor to cancel the association between the patient identification information and the sensor identification information.

5. The mobile information terminal according to claim 1, wherein,
in a case where the wireless communication interface receives the connection request signal from the sensor that is identified by the sensor identification information stored in the storage, when the sensor identification information is not associated with the patient identification information, the processor is configured to cause the wireless communication interface to transmit information indicating denial of establishment of the wireless connection, to the sensor.

6. The mobile information terminal according to claim 1, further comprising: a displaying section that is configured to display the physiological information based on the sensor signal.

7. A physiological information management method that, in a mobile information terminal including a storage, a wireless communication interface, and a processor, is executed by the processor, physiological information management method comprising:
storing, in the storage, sensor identification information for identifying a sensor which is attached to a patient and which wirelessly transmits a sensor signal corresponding to physiological information of the patient, and patient identification information for identifying the patient while associating with each other;
when the wireless communication interface receives a connection request signal from the sensor identified by the sensor identification information stored in the storage, determining whether the sensor identification information is associated with the patient identification information or not;
denying establishment of a wireless connection with the sensor that is subjected to the determination, in a case where the sensor identification information that is subjected to the determination is not associated with the patient identification information;
establishing a wireless connection with the sensor that is subjected to the determination, in a case where the sensor identification information that is subjected to the determination is associated with the patient identification information; and
receiving, by the wireless communication interface, the sensor signal transmitted from the sensor with which the wireless connection is established.

8. A non-transitory computer readable storage medium storing a computer program causing the mobile information terminal to execute the physiological information management method according to claim 7.
